(19) United States
(12) Patent Application Publication
(10) Pub. No.: US 2016/0115556 A1
(43) Pub. Date: Apr. 28, 2016

(54) DETECTING MUTATIONS IN DISEASE OVER TIME

(71) Applicant: Trovagene, Inc., San Diego, CA (US)

(72) Inventors: Mark G. Erlander, Carlsbad, CA (US); Karena Kosco, San Diego, CA (US); Cecile Rose Vibat, San Diego, CA (US)

(73) Assignee: Trovagene, Inc., San Diego, CA (US)

(21) Appl. No.: 14/986,669

(22) Filed: Jan. 2, 2016

Related U.S. Application Data
(63) Continuation-in-part of application No. 14/517,878, filed on Oct. 19, 2014.
(60) Provisional application No. 61/893,216, filed on Oct. 19, 2013.
(60) Provisional application No. 61/977,085, filed on Apr. 8, 2014.
(60) Provisional application No. 61/977,609, filed on Apr. 9, 2014.
(60) Provisional application No. 62/040,363, filed on Aug. 21, 2014.

(51) Int. Cl. C12Q 1/68

(52) U.S. Cl. CPC C12Q 1/6886; C12Q 2600/106; C12Q 2600/156

(57) ABSTRACT

Provided is a method determining responsiveness to a treatment in a patient with a cancer.

### CROSS-REFERENCE TO RELATED APPLICATIONS

This application is a continuation-in-part of application Ser. No. 14/517,878, filed Oct. 19, 2014, which claims the benefit of U.S. Provisional Application No. 61/893,216, filed Oct. 19, 2013, U.S. Provisional Application No. 61/977,085, filed Apr. 8, 2014, U.S. Provisional Application No. 61/977,609, filed Apr. 9, 2014, and U.S. Provisional Application No. 62/040,363, filed Aug. 21, 2014, all of which are incorporated by reference herein in their entirety.

### BACKGROUND OF THE INVENTION

(1) Field of the Invention

The present invention generally relates to cancer mutations. More specifically, the invention provides methods for monitoring cancer mutations over time, which is useful for evaluating treatment options.

(2) Description of the Related Art

Nucleic acids in cancerous tissues, circulating cells, and cell-free (cf) nucleic acids present in bodily fluids can aid in identifying and selecting individuals with cancer or other diseases associated with such genetic alterations. See, e.g., Spindler et al., 2012; Benesova et al., 2013; Dawson et al., 2013; Forshew et al., 2012; Shaw et al., 2012. Some data suggest that the amount of mutant DNA in blood correlates with tumor burden and can be used to identify the emergence of resistant mutations (Forshew et al., 2012; Murtaza et al., 2013; Dawson et al., 2013; Diaz et al., 2012; Misale et al., 2012; Diehl et al., 2008). However, it is unknown whether quantitative or semi-quantitative measurements of cfDNA in blood or urine reflect tumor burden accurately enough to utilize in making treatment decisions.

There is a need for additional non-invasive methods of determining effectiveness of treatment by monitoring tumor burden over time. The present invention addresses that need.

### BRIEF SUMMARY OF THE INVENTION

The present invention is based in part on the discovery that cancer treatment can be monitored by measuring cfDNA in urine or blood at various time points over the course of the treatment.

Thus, in some embodiments, a method of determining responsiveness to a treatment, or overall survival, in a patient with a cancer undergoing the treatment is provided, where the cancer is associated with a gene mutation. The method comprises

(a) obtaining a baseline sample of a bodily fluid, wherein the baseline sample was taken from the patient at the start of the treatment;

(b) quantitatively or semi-quantitatively determining the amount of the mutation in cell free DNA (cfDNA) in the baseline sample;

(c) obtaining a second sample of the bodily fluid, wherein the second sample was taken from the patient within about one month after the start of the treatment;

(d) quantitatively or semi-quantitatively determining the amount of the mutation in cell free DNA (cfDNA) in the second sample; and

(e) determining the responsiveness to the treatment by evaluating the results obtained in step (b) and/or step (d) by a predetermined criteria.

### BRIEF DESCRIPTION OF THE DRAWINGS

FIG. 1 illustrates an exemplary two-step assay design for a 28-30 bp footprint in a target gene sequence.

FIG. 2 are graphs of experimental results showing positive and negative controls for the identification of a BRAF V600E mutation.

FIG. 3 is a graph showing results of BRAF V600E monitoring of a metastatic melanoma patient before treatment, during treatment, and after treatment. No significant recurrence of disease is observed.

FIG. 4 is a graph showing results of BRAF V600E monitoring of a metastatic colorectal cancer patient before treatment, during treatment, and after treatment. Recurrence of disease is observed.

FIG. 5 is a graph showing results of BRAF V600E monitoring of a patient with appendiceal cancer before treatment and during treatment.

FIG. 6 is a graph showing results of BRAF V600E monitoring of a metastatic non-small cell lung cancer patient during treatment. Resistance to the therapy is observed.

FIG. 7 is a graph showing results of BRAF V600E monitoring of an untreated metastatic non-small cell lung cancer patient. Disease progression is observed.

FIG. 8 is a diagram of experimental results showing high concordance of KRAS status between urine, plasma and tissue samples of advanced colorectal cancer patients.

FIG. 9 is a diagram of experimental results showing the monitoring of cfDNA containing the BRAF V600E mutation in relation to response to treatment or therapy of metastatic cancer patients. ctDNA indicates “circulating tumor DNA” that is present in cfDNA.

FIG. 10 is a diagram showing the study design of the study described in Example 7.

FIG. 11 shows Kaplan-Meier survival plots for males, age <65, receiving gemcitabine, with baseline KRAS counts above and below 5.5 copies/10⁵ geq.

FIG. 12 shows Kaplan-Meier survival plots for males, age <65, receiving gemcitabine, with baseline CA 19-9 counts above and below 315 and baseline KRAS counts of ≦5.5 cps/10⁵ geq and >5.5 cps/10⁵ geq.

FIG. 13A and FIG. 13B are plots of KRAS levels after two weeks of treatment for two patients.

### DETAILED DESCRIPTION OF THE INVENTION

As used herein, the singular forms “a”, “an” and “the” are intended to include the plural forms as well, unless the context clearly indicates otherwise. Additionally, the use of “or” is intended to include “and/or” unless the context clearly indicates otherwise.

As used herein, the term “sample” refers to anything which may contain an analyte for which an analyte assay is desired. In many cases, the analyte is a cf nucleic acid molecule, such as a DNA or cDNA molecule encoding all or part of BRAF. The sample may be a biological sample, such as a biological fluid or a biological tissue. Examples of biological fluids include urine, blood, plasma, serum, saliva, semen, stool, sputum, cerebrospinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, a “patient” includes a mammal. The mammal can be e.g., any mammal, e.g., a human, primate, bird, mouse, rat, fowl, dog, cat, cow, horse, goat, camel, sheep or a pig. In many cases, the mammal is a human being.

The present invention is based in part on the discovery that responsiveness to a cancer treatment, or overall survival, can be determined by quantitatively or semi-quantitatively measuring a gene mutation associated with the cancer by measuring nucleic acids, for example cell free (cf) DNA or circulating tumor (ct) DNA, in a bodily fluid at various time points over the course of the treatment. This determination can be made long (weeks or months) before such determinations can be made by conventional means, e.g., radiologically.

Thus, in some embodiments, a method of determining responsiveness to a treatment, or overall survival, in a patient with a cancer undergoing the treatment is provided, where the cancer is associated with a gene mutation. The method comprises

(a) obtaining a baseline sample of a bodily fluid, wherein the baseline sample was taken from the patient at the start of the treatment;

(b) quantitatively or semi-quantitatively determining the amount of the mutation in cell free DNA (cfDNA) in the baseline sample;

(c) obtaining a second sample of the bodily fluid, wherein the second sample was taken from the patient within about one month after the start of the treatment;

(d) quantitatively or semi-quantitatively determining the amount of the mutation in cell free DNA (cfDNA) in the second sample; and

(e) determining the responsiveness to the treatment by evaluating the results obtained in step (b) and/or step (d) by a predetermined criteria.

The steps (a)-(e) need not be performed in that order. For example, the baseline sample and second sample could be obtained (steps (a) and (c)) before the respective determining steps (steps (b) and (d)).

The second sample can be taken from the patient any time within about one month after the start of the treatment, for example, four weeks, three weeks, two weeks, one week, less than one week, or any time in between. In some embodiments, the second sample is taken from the patient at about two weeks after the start of the treatment.

As shown in the Examples, determination of the mutant levels in the baseline sample and in a second sample taken at that time, allows an accurate determination of the responsiveness to the treatment. This allows for the determination of responsiveness much earlier than can be made using more conventional methods, for example by visualizing tumor shrinkage using radiography, computed tomography (CT) scanning, positron emission tomography (PET), or PET/CT scanning. In various embodiments, a decrease in the mutant levels after two weeks indicates responsiveness.

The rapid determination of responsiveness to treatment can be particularly useful in clinical trials, where the determination of responsiveness provides an early assessment of drug efficiency in a population of patients. This allows a more rapid assessment of the drug efficacy than was previously possible.

As further shown in the Examples, a lower level of the mutation in the baseline sample and/or the second sample indicate a longer overall survival than higher mutation levels. In these embodiments, the predetermined criteria is established by comparing treatment outcomes with the amount of the mutation in baseline samples and/or second samples in previous cancer patients.

Any bodily fluid that would be expected to have DNA can be utilized in these methods. Non-limiting examples of bodily fluids include, but are not limited to, peripheral blood, serum, plasma, urine, lymph fluid, amniotic fluid, and cerebrospinal fluid. In certain particular embodiments, such as those illustrated in the Examples, the bodily fluid is serum, plasma or urine.

In some cases, the method is performed quantitatively, such that the amount of the gene alteration is quantitatively determined and may be quantitatively compared to another measurement. In other cases, the method is performed semi-quantitatively, such that the amount of the gene alteration may be determined and then compared to another measurement simply to determine a relative increase or decrease relative to each other.

These methods are not narrowly limited to any particular gene mutations in any particular cancer, since levels of any mutation that is associated with any cancer would be expected to be accurately determined by these methods. Nonlimiting examples of such genes are an ABL1, BRAF, CHEK1, FANCC, GATA3, JAK2, MITF, PDCD1LG2, RBM10, STAT4, ABL2, BRCA1, CHEK2, FANCD2, GATA4, JAK3, MLH1, PDGFRA, RET, STK11, ACVR1B, BRCA2, CIC, FANCE, GATA6, JUN, MPL, PDGFRB, RICTOR, SUFU, AKT1, BRD4, CREBBP, FANCF, GID4(C17orf39), KAT6A (MYST3), MRE11A, PDK1, RNF43, SYK, AKT2, BRIP1, CRKL, FANCG, GLI1, KDM5A, MSH2, PIK3C2B, ROS1, TAF1, AKT3, BTG1, CRLF2, FANCL, GNA11, KDM5C, MSH6, PIK3CA, RPTOR, TBX3, ALK, BTK, CSF1R, FAS, GNA13, KDM6A, MTOR, PIK3CB, RUNX1, TERC, AMER1 (FAM123B), C11orf30 (EMSY), CTCF, FAT1, GNAQ, KDR, MUTYH, PIK3CG, RUNX1T1, TERT promoter, APC, CARD11, CTNNA1, FBXW7, GNAS, KEAP1, MYC, PIK3R1, SDHA, TET2, AR, CBFB, CTNNB1, FGF10, GPR124, KEL, MYCL (MYCL1), PIK3R2, SDHB, TGFBR2, ARAF, CBL, CUL3, FGF14, GRIN2A, KIT, MYCN, PLCG2, SDHC, TNFAIP3, ARFRP1, CCND1, CYLD, FGF19, GRM,3 KLHL6, MYD88, PMS2, SDHD, TNFRSF14, ARID1A, CCND2, DAXX, FGF23, GSK3B, KMT2A (MLL), NF1, POLD1, SETD2, TOP1, ARID1B, CCND3, DDR2, FGF3, H3F3A, KMT2C (MLL3), NF2, POLE, SF3B1, TOP2A, ARID2, CCNE1, DICER1, FGF4, HGF, KMT2D (MLL2), NFE2L2, PPP2R1A, SLIT2, TP53, ASXL1, CD274, DNMT3A, FGF6, HNF1A, KRAS, NFKBIA, PRDM1, SMAD2, TSC1, ATM, CD79A, DOT1L, FGFR1, HRAS, LMO1, NKX2-1, PREX2, SMAD3, TSC2, ATR, CD79B, EGFR, FGFR2, HSD3B1, LRP1B, NOTCH1, PRKAR1A, SMAD4, TSHR, ATRX, CDC73, EP300, FGFR3, HSP9OAA1, LYN, NOTCH2, PRKCI, SMARCA4, U2AF1, AURKA, CDH1, EPHA3, FGFR4, IDH1, LZTR1, NOTCH3, PRKDC, SMARCB1, VEGFA, AURKB, CDK12, EPHA5, FH, IDH2, MAGI2, NPM1, PRSS8, SMO, VHL, AXIN1, CDK4, EPHA7, FLCN, IGF1R, MAP2K1, NRAS, PTCH1, SNCAIP, WISP3, AXL, CDK6, EPHB1, FLT1, IGF2, MAP2K2, NSD1, PTEN, SOCS1, WT1, BAP1, CDK8, ERBB2, FLT3, IKBKE, MAP2K4, NTRK1, PTPN11, SOX10, XPO1, BARD1, CDKN1A, ERBB3, FLT4, IKZF1, MAP3K1, NTRK2, QKI, SOX2, ZBTB2, BCL2, CDKN1B, ERBB4, FOXL2, IL7R, MCL1, NTRK3, RAC1, SOX9, ZNF217, BCL2L1, CDKN2A, ERG, FOXP1, INHBA, MDM2, NUP93, RAD50, SPEN, ZNF703, BCL2L2, CDKN2B, ERRFI1, FRS2, INPP4B, MDM4, PAK3, RAD51, SPOP, BCL6, CDKN2C, ESR1, FUBP1, IRF2, MED12, PALB2, RAF1, SPTA1, BCOR, CEBPA, EZH2, GABRA6, IRF4, MEF2B, PARK2, RANBP2, SRC, BCORL1, CHD2, FAM46C, GATA1, IRS2, MEN1, PAX5, RARA, STAG2, BLM, CHD4, FANCA, GATA2, JAK1, MET, PBRM1, RB1, or STAT3 gene.

In some embodiments, the mutation is in a BRAF gene or a KRAS gene. Exemplary mutations in those genes are BRAF V600E and the KRAS mutations G12A, G12C, G12D, G12R, G12S, G12V and G13D.

An association with BRAF V600E has been reported for various human neoplasms, including melanomas (−50%) (Davies et al., 2002; Curtin et al., 2005), papillary thyroid carcinomas (−40%) (Puxeddu et al., 2004), Langherans cell histiocytosis (57%) (Badalian-Very et al., 2010) and a variety of solid tumors (at lower frequency)(Davies et al., 2002; Brose et al., 2002; Tie et al., 2011).

A member of the serine/threonine kinase RAF family, the BRAF protein is part of the RAS-RAF-MAPK signaling pathway that plays a major role in regulating cell survival, proliferation and differentiation (Keshet and Seger, 2010). BRAF mutations constitutively activate the MEK-ERK pathway, leading to enhanced cell proliferation, survival and ultimately, neoplastic transformation (Wellbrock and Hurlstone, 2010; Niault and Baccarini, 2010). All BRAF mutated hairy cell leukemia (HCL) cases carried the V600E phospho-mimetic substitution which occurs within the BRAF activation segment and markedly enhances its kinase activity in a constitutive manner (Wan et al., 2004).

Non-limiting examples of cancer include, but are not limited to, adrenal cortical cancer, anal cancer, bile duct cancer, bladder cancer, bone cancer, brain or a nervous system cancer, breast cancer, cervical cancer, colon cancer, rectal cancer, colorectal cancer, endometrial cancer, esophageal cancer, Ewing family of tumor, eye cancer, gallbladder cancer, gastrointestinal carcinoid cancer, gastrointestinal stromal cancer, Hodgkin Disease, intestinal cancer, Kaposi Sarcoma, kidney cancer, large intestine cancer, laryngeal cancer, hypopharyngeal cancer, laryngeal and hypopharyngeal cancer, leukemia, acute lymphocytic leukemia (ALL), acute myeloid leukemia (AML), chronic lymphocytic leukemia (CLL), chronic myeloid leukemia (CML), chronic myelomonocytic leukemia (CMML), non-HCL lymphoid malignancy (hairy cell variant, splenic marginal zone lymphoma (SMZL), splenic diffuse red pulp small B-cell lymphoma (SDRPSBCL), chronic lymphocytic leukemia (CLL), prolymphocytic leukemia, low grade lymphoma, systemic mastocytosis, or splenic lymphoma/leukemia unclassifiable (SLLU)), liver cancer, lung cancer, non-small cell lung cancer, small cell lung cancer, lung carcinoid tumor, lymphoma, lymphoma of the skin, malignant mesothelioma, multiple myeloma, nasal cavity cancer, paranasal sinus cancer, nasal cavity and paranasal sinus cancer, nasopharyngeal cancer, neuroblastoma, non-Hodgkin lymphoma, oral cavity cancer, oropharyngeal cancer, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma, adult soft tissue sarcoma, skin cancer, basal cell skin cancer, squamous cell skin cancer, basal and squamous cell skin cancer, melanoma, stomach cancer, small intestine cancer, testicular cancer, thymus cancer, thyroid cancer, uterine sarcoma, uterine cancer, vaginal cancer, vulvar cancer, Waldenstrom Macroglobulinemia, and Wilms Tumor.

Non-limiting examples of non-HCL lymphoid malignancy include, but are not limited to, hairy cell variant (HCL-v), splenic marginal zone lymphoma (SMZL), splenic diffuse red pulp small B-cell lymphoma (SDRPSBCL), splenic leukemia/lymphoma unclassifiable (SLLU), chronic lymphocytic leukemia (CLL), prolymphocytic leukemia, low grade lymphoma, systemic mastocytosis, and splenic lymphoma/leukemia unclassifiable (SLLU).

In certain embodiments of these methods, overall survival is determined in a patient with pancreatic cancer, for example where the gene mutation is a KRAS codon 12 or codon 13 mutation. As shown in Example 7, overall survival determination is improved by determining CA 19-9 antigen levels. In various embodiments, results from both the baseline sample and the second sample are evaluated.

In other embodiments of these methods, responsiveness to treatment for colorectal cancer is determined by evaluating results from both the baseline sample and the second sample. In these embodiments, a reduction in levels of the mutation indicates responsiveness. In some embodiments, the colorectal cancer is metastatic colorectal cancer; in various embodiments, the gene mutation is a KRAS codon 12 or 13 mutation.

In various embodiments of the methods described herein, the patients are humans. The patients may be of any age, including, but not limited to infants, toddlers, children, minors, adults, seniors, and elderly individuals.

In any of the methods described herein, the mutation can be determined, or quantified, by any method known in the art. Nonlimiting examples include MALDI-TOF, HR-melting, di-deoxy-sequencing, single-molecule sequencing, use of probes, pyrosequencing, second generation high-throughput sequencing, SSCP, RFLP, dHPLC, CCM, or methods utilizing the polymerase chain reaction (PCR), e.g., digital PCR, quantitative-PCR, or allele-specific PCR (where the primer or probe is complementary to the variable gene sequence). In some embodiments, the PCR is droplet digital PCR, e.g., as described in the Examples. In some of these methods, the mutation is quantified along with the wildtype sequence, to determine the percentage of mutated sequence. In other methods, only the mutation is quantified.

In many embodiments, the DNA is cell free DNA (“cfDNA”), or circulating tumor DNA (ctDNA), which may include DNA from cells. In some embodiments, the amplified or detected DNA molecule is genomic DNA. In other embodiments, the amplified or detected molecule is a cDNA.

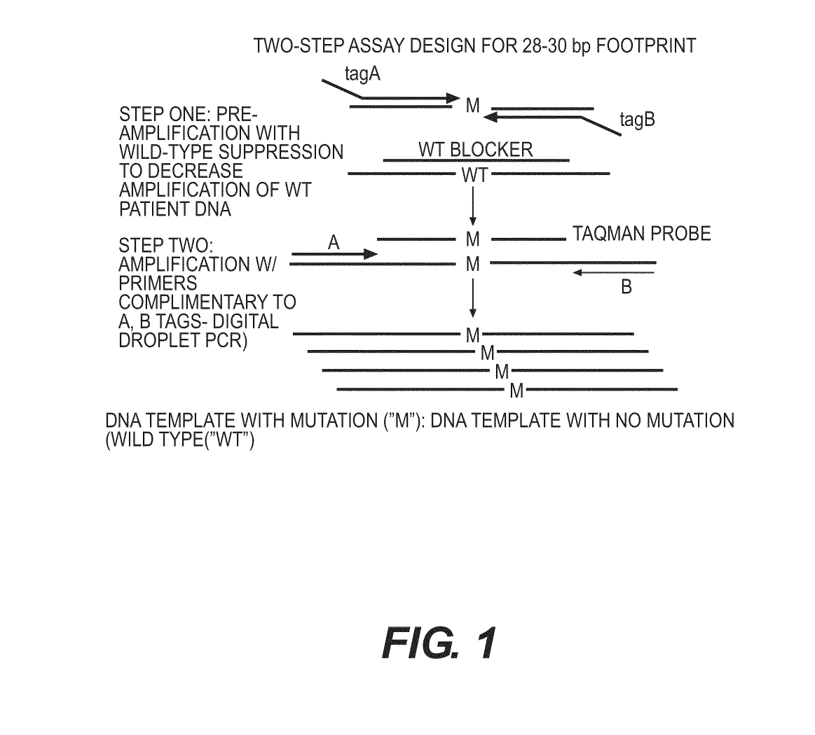

The skilled artisan can determine useful primers for PCR amplification of any mutant sequence for any of the methods described herein. In some embodiments, the PCR amplifies a sequence of less than about 50 nucleotides, e.g., as described in US Patent Application Publication US/2010/0068711. In other embodiments, the PCR is performed using a blocking oligonucleotide that suppresses amplification of a wildtype version of the gene, e.g., as illustrated in FIG. 1 (see also Example 1 below) or as described in U.S. Pat. No. 8,623,603 or U.S. Provisional Patent Application No. 62/039,905. In many embodiments, one or more primers contains an exogenous or heterologous sequence (such as an adapter or “tag” sequence), as is known in the art, such that the resulting amplified molecule has a sequence that is not naturally occurring.

The detection limits for the presence of a gene alteration (mutation) in cf nucleic acids may be determined by assessing data from one or more negative controls (e.g. from healthy control subjects or verified cell lines) and a plurality of patient samples. Optionally, the limits may be determined based in part on minimizing the percentage of false negatives as being more important than minimizing false positives. One set of non-limiting thresholds for is defined as less than about 0.05% of the mutation in a sample of cf nucleic acids for a determination of no mutant present or wild-type only; the range of about 0.05% to about 0.107% as “borderline”, and greater than about 0.107% as detected mutation. In other embodiments, a no-detection designation threshold for the mutation is set at less than about 0.1%, less than about 0.15%, less than about 0.2%, less than about 0.3%, less than about 0.4%, less than about 0.5%, less than about 0.6%, less than about 0.7%, less than about 0.8%, less than about 0.9%, or less than about 1% detection of the mutation relative to a corresponding wildtype sequence.

A borderline designation can also be set according to any criteria, including the relative amount of false positives and false negatives desired.

Of course the inclusion of additional patient samples may result in the determination of different threshold values for each category, or alternatively the elimination of the “borderline” category. The desired amount of false negatives to false positives will also have an effect on the threshold value.

The “obtaining” and “determining” steps of these methods can be repeated as many times as necessary to obtain sufficient data to assist in determining treatment options, overall survival, or the effectiveness of the treatment being applied. In some embodiments, these steps are performed weekly, monthly, every two months, every three months, every four months, or any interval in between those time points.

In some embodiments, the patient has not previously undergone testing for the mutation in the gene. In those situations, the method are used to determine whether a specific mutation is involved in the cancer, and whether a medicament that targets the product of the gene having the mutation could be effective. For example, where a BRAF V600E mutation is present, the patient might be treated with a BRAF inhibitor such as vemurafenib, sorafenib or dabrafenib.

In some embodiments, the patient has been previously tested and a mutation determined, and the subsequent tests are to evaluate the progression of the disease and/or the effectiveness of treatment. In some cases, the detecting may identify the non-responsiveness to a treatment or therapy, and the selecting and/or applying comprises a different treatment or therapy. In other cases, the detecting may identify the responsiveness to a treatment or therapy, and the selecting and/or applying comprises continuation of the same treatment or therapy. In additional embodiments, the monitoring is a surveillance of patients, e.g., treated patients deemed “disease free” where there is a chance of recurrence.

Thus, these methods may be used to confirm the maintenance of a disclosed treatment or therapy against various diseases including cancer; or to change the treatment or therapy against the disease. In that context, a method of selecting and/or applying treatment or therapy for a subject is also provided herein. The method comprises monitoring a gene mutation by the above method, and selecting and/or applying a treatment or therapy based on the detecting.

In some embodiments of these methods, the method identifies low responsiveness or non-responsiveness to a treatment or therapy, and the selecting and/or applying comprises a different treatment or therapy. In other embodiments, the method identifies effective treatment or therapy, and the selecting and/or applying comprises continuing the same treatment or therapy. In additional embodiments, the method identifies elimination of the mutation and the selecting and/or applying comprises discontinuing treatment.

Within the scope of changing treatment or therapy, the disclosure includes increasing the treatment or therapy; reducing the treatment or therapy, optionally to the point of terminating the treatment or therapy; terminating the treatment or therapy with the start of another treatment or therapy; and adjusting the treatment or therapy as non-limiting examples. Non-limiting examples of adjusting the treatment or therapy include reducing or increasing the therapy, optionally in combination with one or more additional treatments or therapies; or maintaining the treatment or therapy while adding one or more additional treatments or therapies.

In some cases, the observation of cell-free (cf) nucleic acids identifies an increase in the levels of cf nucleic acids containing the mutation following the start of a treatment or therapy. Following the increase, the observation may reach an inflection point, where the levels decrease, or continue to increase. The presence of an inflection point may be used to determine responsiveness to the treatment or therapy, which may be maintained or reduced. A continuing decrease in the levels to be the same as, or lower than, the levels before the start of treatment of therapy is a further confirmation of responsiveness.

The absence of an inflection point indicates resistance to the treatment or therapy and so may be followed by terminating administration of the treatment or therapy, or administering at least one additional treatment or therapy against the disease or disorder to the patient, reducing the treatment of the subject with the treatment or therapy and administering at least one additional treatment or therapy against the disease or disorder to the subject.

In other cases, and following an inflection point and a decrease in levels, an additional inflection point may be observed. This may indicate the development of resistance to the treatment or therapy and be followed by terminating administration of the treatment or therapy, or administering at least one additional treatment or therapy against the disease or disorder to the subject, or reducing the treatment of the subject with the therapy and administering at least one additional therapy against the disease or disorder to the subject.

In some aspects, the method is accompanied by a conventional determination of the tumor burden, e.g., by radiography, computed tomography (CT) scanning, positron emission tomography (PET), or PET/CT scanning, and comparing the determined amount of mutation to the tumor burden. This is useful to determine whether, or confirm that, the mutation being monitored is actually the driver of the tumor.

In other aspects, the determined amount of mutation is not compared to tumor burden, either at one, more than one, or all the mutation monitoring times. Given the reliability of the mutation monitoring procedures described herein, a tumor burden assessment need not be made at each time point, thus saving the patient a tumor burden assessment.

In additional aspects, the monitoring comprises evaluating a mutation that is associated with a time-to-failure parameter (i.e., the treatment directed to the mutation is known to fail after a certain period of effectiveness). In these aspects, the monitoring can assist in more accurately predicting when failure will occur, for example when the concentration of the mutation increases over a previous assessment.

Treatments and therapies of the disclosure include all modalities of cancer therapy. Non-limiting examples of these modalities include radiation therapy, chemotherapy, hormonal therapy, immunotherapy, and surgery. Non-limiting examples of radiation therapy include external beam radiation therapy, such as with photons (gamma radiation), electrons, or protons; stereotactic radiation therapy, such as with a single high dose or multiple fractionated doses to a small target; brachytherapy; and systemic radioactive isotopes.

Non-limiting examples of chemotherapy include cytotoxic drugs; antimetabolites, such as folate antagonists, purine antagonists, and pyrimidine antagonists; biological response modifiers, such as interferons; DNA damaging agents, such as bleomycin; DNA alkylating and cross-linking agents, such as nitrosourea and bendamustine; enzymatic activities, such as asparaginase; hormone antagonists, such as fulvestrant and tamoxifen; aromatase inhibitors; monoclonal antibodies; antibiotics such as mitomycin; platinum complexes such as cisplatin and carboplatin; proteasome inhibitors such as bortezomib; spindle poison such as taxanes or vincas or derivatives of either; topoisomerase I and II inhibitors, such as anthracyclines, camptothecins, and podophyllotoxins; tyrosine kinase inhibitors; anti-angiogenesis drugs; and signal transduction inhibitors.

Non-limiting examples of hormonal therapy include hormone antagonist therapy, hormone ablation, bicalutamide, enzalutamide, tamoxifen, letrozole, abiraterone, prednisone, or other glucocorticosteroid. Non-limiting examples of immunotherapy include anti-cancer vaccines and modified lymphocytes.

In some cases, the maintenance of, or change in, treatment or therapy is within one of these modalities. In other cases, the maintenance of, or change in, treatment or therapy is between two or more of these modalities. Of course a skilled clinician is aware of the recognized and approved treatments and therapies for a given disorder or disease, such as a particular cancer or tumor type, and so the maintenance of, or change in, treatment or therapy may be within those known for the disease or disorder.

The present disclosure also provides, in part, a kit for performing the disclosed methods. The kit may include a specific binding agent that selectively binds to a BRAF mutation, and instructions for carrying out the method as described herein.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3rd edition), Cold Spring Harbor Press, Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18th edition (1990). These texts can, of course, also be referred to in making or using an aspect of the disclosure.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLES

### Example 1

### Materials and Methods

The following methods were utilized in the examples that follow.

### Patient Urine Samples

A total of 27 patients with metastasized cancers, whose tumor samples were previously tested for mutations in BRAF (20 patients) and KRAS (7 patients) by a CLIA-certified laboratory, were prospectively enrolled.

Single or multiple sequential urine samples (90-110 ml or 24 hour urine collection) for cfDNA mutation analysis were obtained at baseline and during therapy and post-therapy.

### Two-Step Assay Design

A two-step assay design was developed for a 28-30 basepair footprint in the target mutant gene sequence. This assay design (and other assays known in the art) is useful for amplifying any size sequence in various tissues or bodily fluids, for example less than 400, less than 300, less than 200, less than 150 bp, less than 100 bp, less than 50 bp, less than 40 bp, less than 35 bp, or less than 30 bp.

FIG. 1 summarizes the assay design, which includes a first pre-amplification step to increase the number of copies of a target mutant gene sequence relative to wild-type gene sequences that are present in the sample. The pre-amplification is conducted in the presence of a wild-type (non-mutant) suppressing “WT blocker” oligonucleotide that is complementary to the wild-type sequence (but not the mutant sequence) to decrease amplification of wild-type DNA. The pre-amplification is performed with primers that include adapters (or “tags”) at the 5′ end to facilitate amplification in the second step.

The second step is additional amplification with primers complementary to the tags on the ends of the primers used in the first step and a TaqMan (reporter) probe oligonucleotide complementary to the mutant sequence for quantitative, digital droplet PCR.

### Assay Development

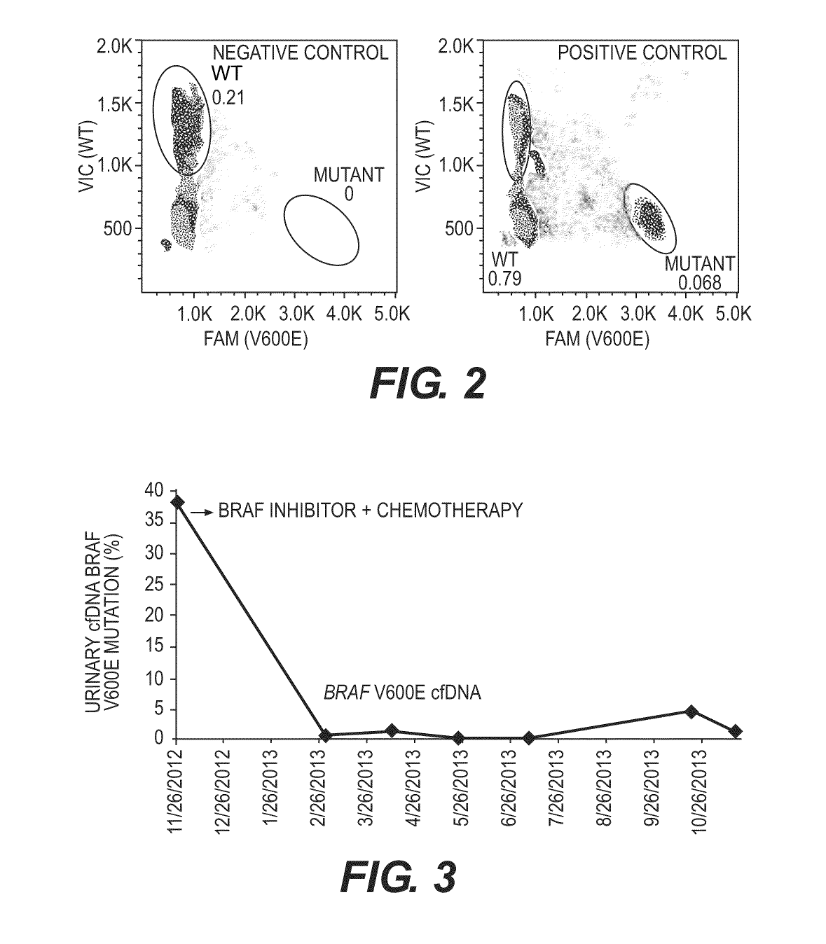

Cell lines with respective mutations (BRAF V600E, KRAS G12D, or KRAS G12V) were used as positive controls. Cell lines confirmed as wildtype BRAF and KRAS were used as negative controls. See FIG. 2.

Thresholds for mutation detection were determined by assessing data from 50 healthy controls and 39 patient samples using a classification tree. Minimizing the percentage of false negatives was given a higher importance than minimizing false positives.

A set of non-limiting thresholds for BRAF V600E were defined: <0.05% as no detection or wild-type; the range of 0.05% to 0.107% as “borderline”, and >0.107% as detected mutation. A count of KRAS G12 mutations per sample was used as a non-limiting means to confirm CLIA-identified G12 healthy (wild-type) and G12 mutation samples: <234 mutant fragments as wild-type; and 489-2825 mutant fragments as detected mutation.

### Example 2

### BRAF V600E Mutations in cfDNA

The sensitivity of the two-step assay was first assessed in urine samples from 19 patients with cancers identified as having a BRAF V600E mutation by a CLIA laboratory. The agreement rate of CLIA V600E to urinary cfDNA V600E mutation and “borderline” was 95% as shown in Table 1.

TABLE 1

| | | Urinary cfDNA BRAF |
|---|---|---|
| Tumor type and patient no. | Tumor (CLIA) | V600E mutation (%)* |
| Non-small cell lung cancer; 15 | V600E | V600E (0.17) |
| Papillary thyroid carcinoma; 19 | V600E | V600E (0.17) |
| Non-small cell lung cancer; 16 | V600E | V600E (1.08) |
| Melanoma; 5 | V600E | V600E (37.9) |
| Non-small cell lung cancer; 13 | V600E | V600E (0.68) |
| Colorectal cancer; 1 | V600E | V600E (21.12) |
| Melanoma; 8 | V600E | V600E (0.13) |
| Colorectal cancer; 3 | V600E | V600E (1.49) |
| Glioblastoma; 19 | V600E | V600E (5.36) |
| Melanoma; 10 | V600E | Borderline V600E (0.07) |
| Melanoma; 11 | V600E | Negative V600E (0.04) |
| Melanoma; 9 | V600E | V600E (0.15) |
| Adenocarcinoma of | V600E | Borderline V600E (0.07) |
| unknown primary; 14 | | |
| Colorectal cancer; 2 | V600E | V600E (416.58) |
| Non-small cell lung cancer; 12 | V600E | V600E (2.93) |
| Melanoma; 7 | V600E | V600E (0.97) |
| Papillary thyroid carcinoma; 18 | V600E | V600E (1.66) |
| Melanoma; 6 | V600E | V600E (1.01) |
| Ovarian cancer; 17 | V600E | Borderline V600E (0.08) |
| Appendiceal cancer; 4 | V600E | V600E (3.43) |

*In patients with several sequential urine collections over time, samples with highest mutant fraction are indicated.

Further concordance of the presence of a BRAF V600E mutation in tissue (by a CLIA laboratory) to urinary cfDNA V600E mutation was observed with both baseline urine samples (before treatment) and any assessed point of urine sample. Those results are provided in Table 2 and 3.

TABLE 2
Concordance of BRAF V600E Tissue (CLIA) to Baseline Urine cfDNA

| Tested (N = 33) | BRAF Mutation Urine | BRAF Wild Type Urine |
|---|---|---|
| BRAF Mutation CLIA | 25 | 7 |
| BRAF Wild Type CLIA | 0 | 0 |

| | |
|---|---|
| Observed Agreements | 25 (76%) |

TABLE 3
Concordance of BRAF V600E Tissue (CLIA) to Any
Assessed Point of Urine cfDNA

| | BRAF | BRAF Wild |
|---|---|---|
| Tested (N-33) | Mutation Urine | Type Urine |
| BRAF Mutation CLIA | 31 | 2 |
| BRAF Wild Type CLIA | 0 | 0 |

| | |
|---|---|
| Observed Agreements | 31 (94%) |

Additionally, cfDNA with the BRAF V600E mutation correlates with its presence in tissue samples from advanced cancer patients, as shown in Table 4. The BRAF V600E mutation was detected in the urine of patients with colorectal, NSCLC (non-small cell lung cancer), ovarian, melanoma, papillary thyroid cancers and other cancers. The disclosed V600E assay demonstrated high concordance in comparison to tissue biopsies (88% detected in urine at any time point tested; 29 of 33 subjects).

TABLE 4

| | | Baseline | Longitudinal |
|---|---|---|---|
| | | Urinary BRAF | Urinary |
| | | V600E cfDNA | BRAF V600E |
| Tumor Type | Tissue (CLIA) | Detection | cfDNA Detection |
| Appendiceal | BRAF V600E | Mutant | Mutant |
| Adenocarcinoma | BRAF V600E | Mutant | Mutant |
| Cholangiocarcinoma | BRAF V600E | Mutant | Mutant |
| Colorectal Cancer | BRAF V600E | Mutant | Mutant |
| Colorectal Cancer | BRAF V600E | Mutant | Mutant |
| Melanoma | BRAF V600E | Mutant | Mutant |
| NSCLC | BRAF V600E | Low Mutant | Mutant |
| NSCLC | BRAF V600E | Mutant | Mutant |
| Papillary Thyroid | BRAF V600E | Low Mutant | Mutant |
| Papillary Thyroid | BRAF V600E | Mutant | Not Done |

### Example 3

### KRAS G12D Mutations in cfDNA

The sensitivity of the two-step assay was also assessed in urine samples from 7 patients with cancers identified as having a KRAS G12D mutation by a CLIA laboratory. The agreement rate of CLIA G12D to urinary cfDNA G12D mutation was 100% as shown in Table 5.

TABLE 5

| | | Baseline G12 |
|---|---|---|
| | Tumor | KRAS-mutant urinary |
| Tumor Type | (CLIA) | cfDNA (mutant fragments) |
| Colorectal Cancer | G12D | G12D (489) |
| Colorectal Cancer | G12D | G12D (563) |
| Colorectal Cancer | G12D | G12D (1935) |
| Colorectal Cancer | G12D | G12D (2825) |
| Colorectal Cancer | G12V | G12D (1168) |
| Non-Small Cell Lung Cancer | G12V | G12D (1083) |
| Appendiceal Cancer | G12D | G12D (1231) |

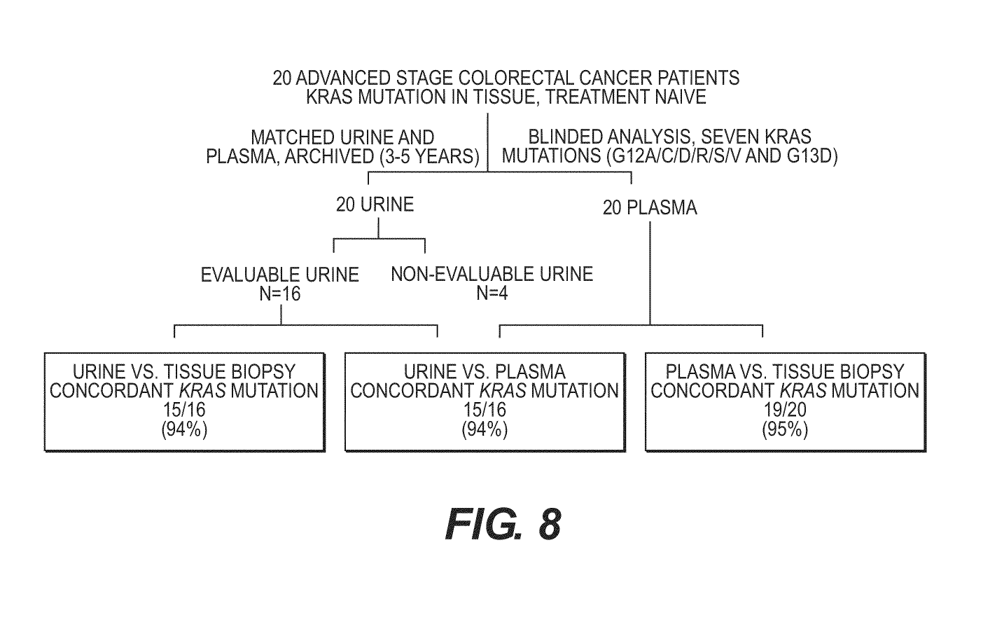

Matched urine and plasma samples that had been archived 3-5 years from 20 advanced stage and treatment naïve colorectal cancer patients were assessed as described herein for the KRAS mutation in comparison to matched tissue samples. The results are shown in FIG. 8, which illustrates the high concordance between all three sample types.

### Example 4

### Longitudinal Assessment of cfDNA Mutations

In three patients a series of multiple urine samples obtained over time was assayed as described above. The patients were afflicted with metastatic melanoma (treated with a BRAF inhibitor and chemotherapy), metastatic colorectal cancer (treated with a BRAF inhibitor and an anti-EGFR antibody), and appendiceal cancer (treated with a BRAF inhibitor and a kinase inhibitor).

The results for the melanoma patient are shown in FIG. 3. A signal of 37.9% was observed in the patient's initial sample, followed by the start of therapy. The subsequent four samples had values of 0.08%, 0.83%, 0.17%, and 0.04%. After termination of treatment, the observed levels of the BRAF V600E mutation in urinary cfDNA remained low.

The results for the colorectal cancer patient are shown in FIG. 4. A signal of 1.49% was observed in the patient's initial sample, followed by the start of therapy. The subsequent four samples had values of 0.09%, 0.00%, 0.00%, and 0.00%. After termination of treatment, the observed levels of the BRAF V600E mutation in urinary cfDNA remained low and then began to increase.

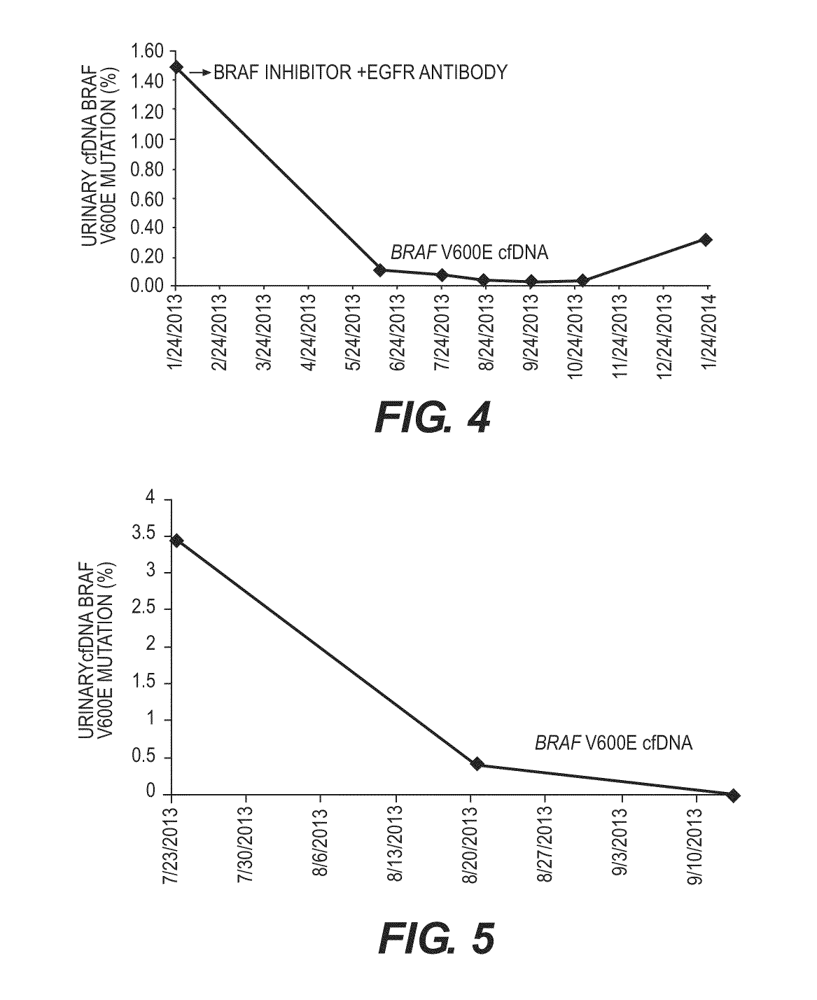

The results for the appendiceal patient are shown in FIG. 5. A signal of 3.43% was observed in the patient's initial sample which was concurrent with therapy. The subsequent two samples had values of 0.45% and 0.02%.

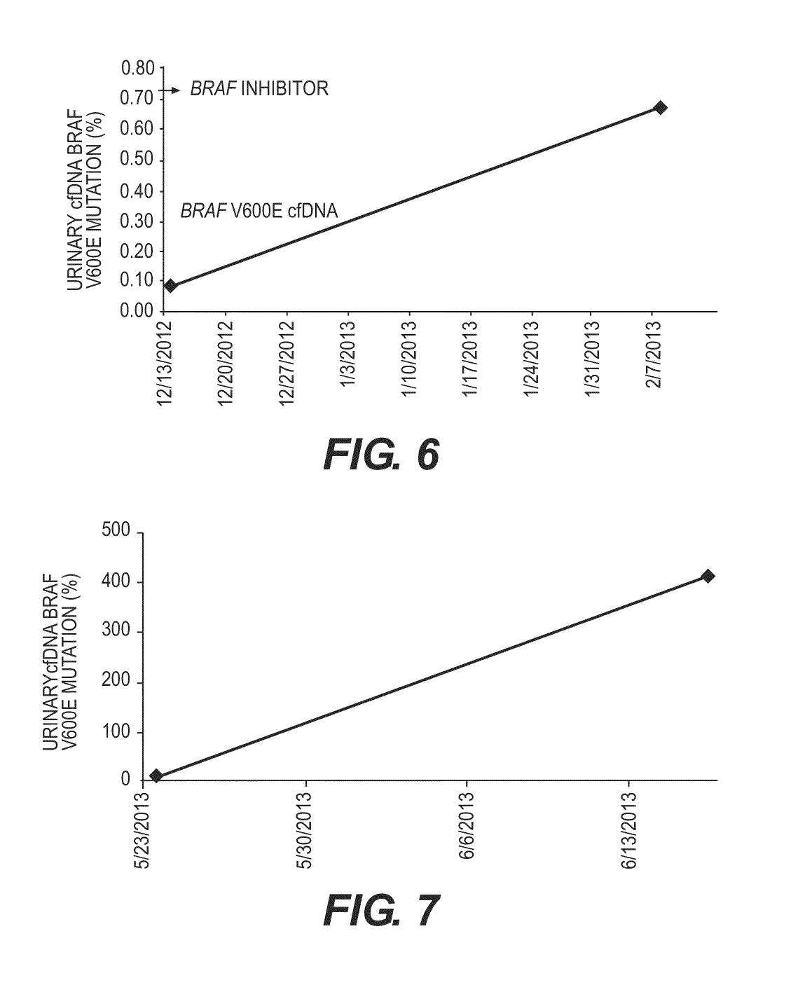

In a fourth and fifth patients with metastatic non-small cell lung cancer, resistance to a BRAF inhibitor was observed during treatment of one patient (FIG. 6). The increase in BRAF V600E mutation in urinary cfDNA urinary was similar to that of an untreated patient (FIG. 7).

In total, longitudinal analysis of BRAF V600E in 17 of 32 metastatic cancer patients was performed by testing serially collected urine. The dynamics of urinary cell-free BRAF V600E correlated with responsiveness (or lack of response) to therapy in 13 of 17 advanced cancer patients (76%).

### Example 5

### Monitoring Presence of BRAF V600E Mutation Vs. Treatment Response

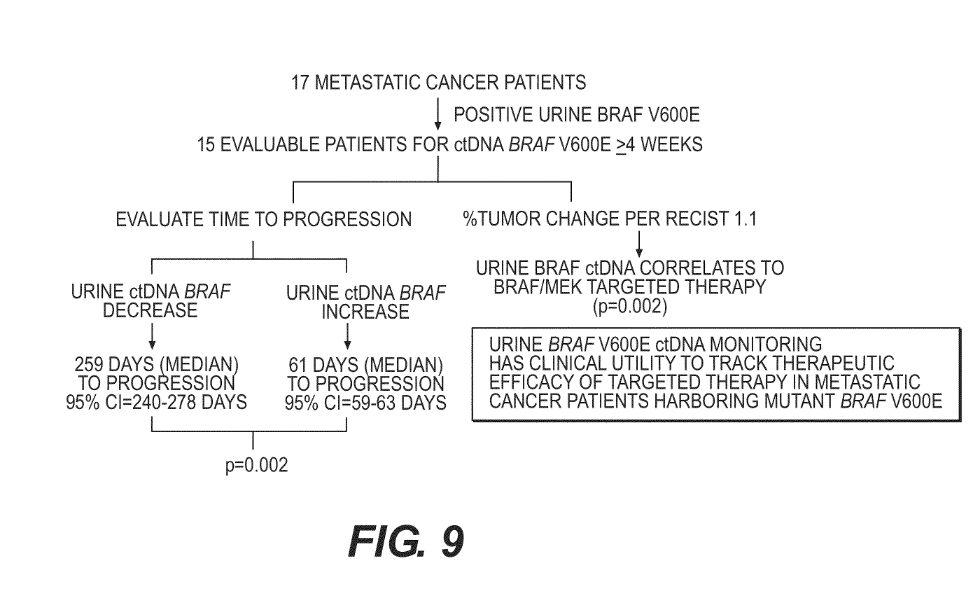

In 15 of 17 metastatic cancer patients that were positive for BRAF V600E cfDNA in urine, the BRAF V600E cfDNA (or ctDNA, circulating tumor DNA) in urine was evaluated over time to monitor disease progression and/or responsiveness to therapy. As shown in FIG. 9, the monitoring has clinical utility for tracking the therapeutic efficacy of targeted therapy in metastatic cancer patients with detectable BRAF V600E cfDNA or ctDNA.

### Example 6

### Monitoring ctDNA KRAS for Response to Chemotherapy

### Example Abstract

Colorectal cancer (CRC) is the third leading cause of cancer mortality in the United States. Despite advances in early detection, each year more than 50,000 patients are diagnosed with metastatic disease. Combination chemotherapy, targeted drugs, and surgical interventions have revolutionized the treatment landscape and improved survival of these patients. Clonal evolution is considered a major cause of drug resistance and non-invasive strategies to detect new and evolving mutations can impact the delivery of personalized treatment. Moreover, non-invasive techniques have the potential to transform the standard of response assessment in metastatic colorectal cancer (mCRC) and reduce the need for imaging in the management of CRC.

### Study Design

This example provides an interim analysis of 4 metastatic CRC patients with positive KRAS tissue status. Three of the patients had metastases in the liver, the fourth patient had metastases in the lung. The patients were monitored for KRAS ctDNA during chemotherapy. All four patients were on FOLFOX treatment; two had surgical intervention.

Urine was collected every two weeks on treatment (x6), and with each radiologic scan (at 6-8 weeks). Urinary ctDNA was extracted using a Trovagene platform that preferentially isolates small fragmented DNA. Highly sensitive, quantitative mutation enrichment PCR-NGS (MiSeq) assay was used for the detection of KRAS codon 12/13 mutations in the highly fragmented ctDNA. For greater sensitivity in fragmented ctDNA, the assay utilizes a 31 bp footprint. A selective enrichment step for mutated DNA fragments suppresses wild-type (WT) sequence amplification with a blocker. Barcoded adaptor primers are added for compatibility with next generation sequencing (MiSeq).

### Results

The dynamics of urinary ctDNA KRAS G12/13 mutational load correlated with clinical course in mCRC patients. A decrease in urine ctDNA KRAS G12/13 mutation levels after 2 weeks of chemotherapy detected the molecular response in advance of radiographic response. In one patient (Patient 1), radiographic progression was detected 3 months after rising ctDNA KRAS mutation was observed in urine.

### Conclusions

Based on the above results, it is clear that the ctDNA KRAS G12/13 assay can be used to guide treatment decisions in metastatic colorectal cancer (mCRC) patients.

### Example 7

### Monitoring KRAS in Pancreatic Cancer

### Background

The overall survival (OS) time of patients with unresectable pancreatic cancer (PC) varies widely. Diagnostic tools are presently lacking to predict patient outcome.

The vast majority of pancreatic tumors harbor KRAS mutations, which can be detected in circulating tumor (ct)DNA.

### Study Aim

The aim of this prospective study with retrospectively analyzed samples was to evaluate the utility of baseline and serial measurements of KRAS mutation load in ctDNA, alone or in combination with CA-19-9, as an outcome predictive marker in locally advanced and metastatic PC patients undergoing palliative chemotherapy.

### Clinical Study Design

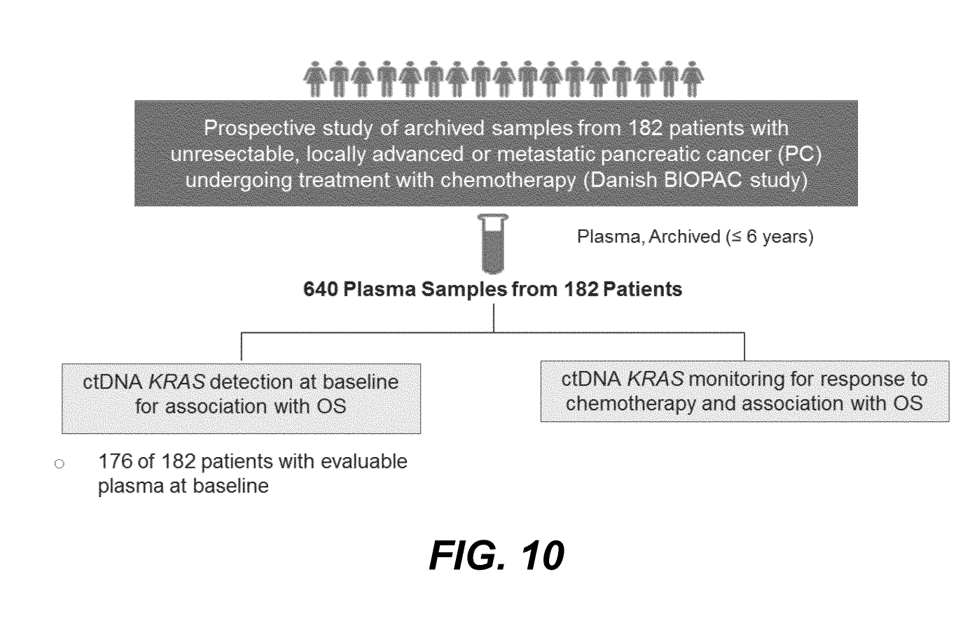

A prospective study of archived samples from 182 patients with non-resectable, locally advanced or metastatic PC undergoing treatment with chemotherapy (Danish BIOPAC study) was performed. FIG. 10 is a graphic of the study design. Patient demographics: 84 females and 92 males, median age 68, range 45-89 years. Locally advanced (n=50) or metastatic (n=132) pancreatic cancer. Palliative treatment was with gemcitabine or FOLFIRINOX.

### Methodology

Highly sensitive, quantitative mutation enrichment PCR-NGS (MiSeq) assay was used for the detection of KRAS codon 12/13 mutations in highly fragmented plasma ctDNA. The Lower Limit of Detection (LLoD) of ctDNA KRAS G12/13 assay is 0.002% mutant alleles in a background of wild-type DNA.

### Results

Table 6 shows the results from this study.

TABLE 6

| Variable | HR (95% CI) | p-value |
|---|---|---|
| Baseline KRAS | 2.4 (1.6-3.4) | <0.0001 |
| ≧5.5 v. <5.5 copies/10⁵ geq | 1.6 (1.2-2.3) | 0.005 |
| CA 19-9 ≧ 315 v. <315 U/mL | 1.6 (1.2-2.2) | 0.004 |
| Gender (male v. female) | 1.6 (0.96-2.5) | 0.075 |
| Chemotherapy (gemcitabine v. FOLFIRINOX) | 1.2 (0.8-1.7) | 0.40 |
| Age (65-75 v. ≦65) | 1.8 (1.1-2.8) | 0.02 |

There was a statistically significant negative association between baseline ctDNA KRAS G12/13 copies and overall survival (OS) in a multivariate COX proportional hazards analysis, indicating that patients with lower systemic KRAS burden survive longer (p<0.0001). Stage was not significant in this analysis.

The hazard ratio (HR) of death for patients with ≧5.5 KRAS copies/10⁵ genome equivalents (GE or geq) is 2.4 times as high (95% CI: 2.0 to 4.9) as those with KRAS G12/13 copies <5. 5/10⁵ GE.

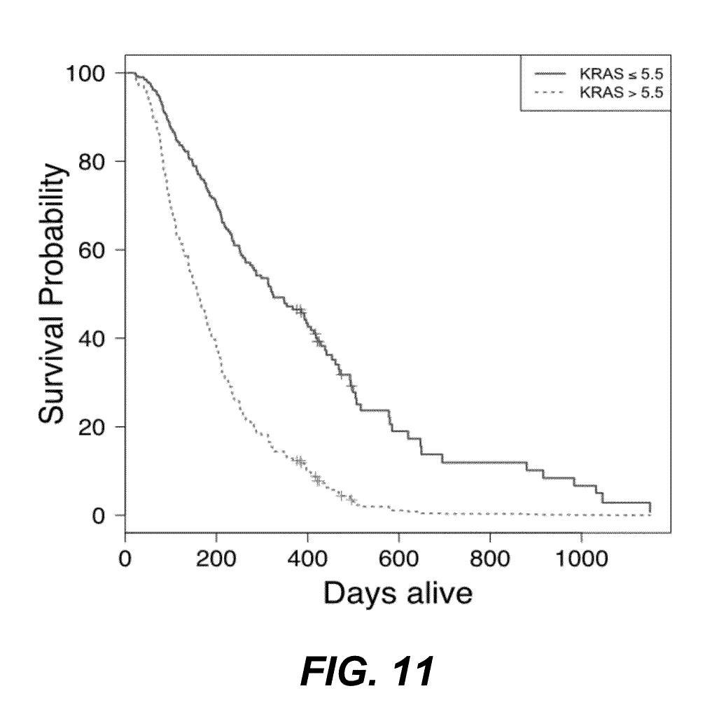

FIG. 11 shows estimated Kaplan-Meier survival plots for males, age <65, receiving gemcitabine, with baseline KRAS counts above and below 5.5 copies/10⁵ geq. Similar results were obtained for female and older patients.

Combination of ctDNA KRAS G12/14 and CA 19-9 Antigen Levels

A combination of pre-treatment levels of ctDNA KRAS G12/13 and CA 19-9 allows for a better fit of the model and a stronger association with OS. R²=23.9%, as compared 19.7% for the model with KRAS alone. Results are shown in Table 7.

TABLE 7

| KRAS copies/10⁵ geq | CA 19-9 U/mL | N | HR (95% CI) |
|---|---|---|---|
| ≦5.5 | <315 | 29 | 1.0 |
| ≧5.5 | ≧315 | 30 | 1.6 (0.94 to 2.8) p = 0.08 |
| >5.5 | <315 | 29 | 2.5 (1.4 to 2.8) p = 0.002 |
| >5.5 | ≧315 | 85 | 4.1 (2.5-6.8) p < 0.0001 |

The HR of death for patients with ≧5.5 KRAS copies/10⁵ GE and ≧315 U/mL CA 19-9 is 4.1 times as high as those with low KRAS and CA 19-9.

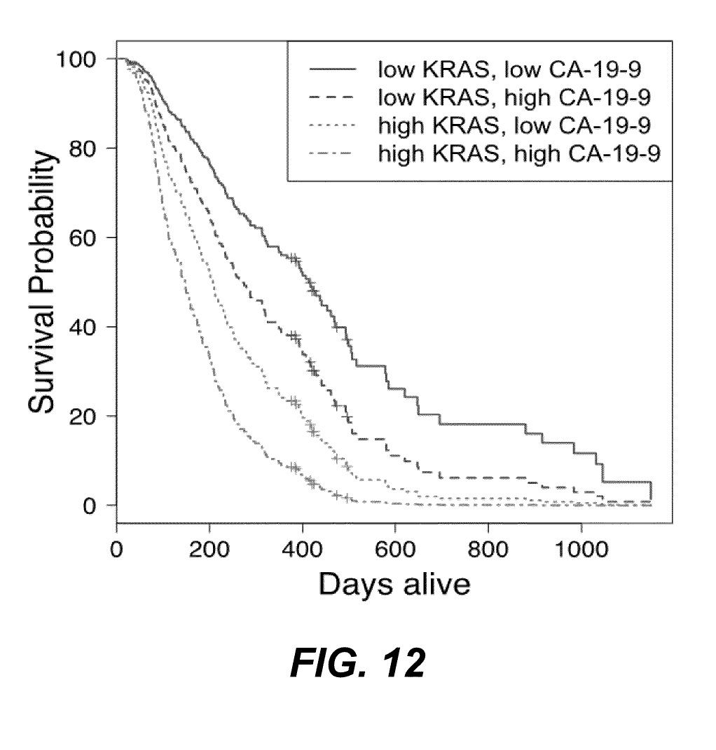

FIG. 12 shows the estimated Kaplan-Meier survival plots for males, age <65, receiving gemcitabine, with baseline CA 19-9 counts above and below 315 and baseline KRAS counts of ≦5.5 cps/10⁵ geq and >5.5 cps/10⁵ geq. Similar results obtained for female and older patients.

Monitoring ctDNA KRAS G12/13 Mutations Longitudinally Post-Treatment is Associated with Outcomes in Patients with Unresectable Pancreatic Cancer

Monitoring ctDNA KRAS levels on therapy may reflect tumor dynamics and better correlate with patient outcomes. In order to account for the effect of therapy, a time-dependent model was built that allows adjustment of estimated patient survival based on the combination of pre-treatment ctDNA KRAS levels and KRAS levels after 2 weeks on first line chemotherapy. Table 8 shows patient characteristics and baseline KRAS pre-treatment levels.

TABLE 8
Patients with Decreases in ctDNA KRAS G12/13 within First
2 Weeks of Chemotherapy Achieve Survival Benefit

| | | | | |
|---|---|---|---|---|
| Baseline KRAS | Week 2 KRAS | | Median | 95% CI for |
| Pre-treatment | End of Cycle 1 | | Overall | Median Overall |
| (cps/10⁵ geq) | (cps/10⁵ geq) | N | Survival | Survival |

Gemcitabine

| | | | | |
|---|---|---|---|---|
| >5.5 | Examining | 103 | 148 days | 127-180 days |
| | baseline only | | | |
| >5.5 | <100% decrease | 78 | 134 days | 104-159 days |
| | from baseline | | | |
| >5.5 | 100% decrease | 20 | 224 days | 189-325 days |
| | from baseline | | | |

FOLFIRINOX

| | | | | |
|---|---|---|---|---|
| >5.5 | Examining | 13 | 210 days | 185-N.E.* days |
| | baseline only | | | |
| >5.5 | <75% decrease from | 6 | 194 days | 94-N.E. days |
| | baseline | | | |
| >5.5 | ≧75% decrease from | 4 | 309 days | 209-N.E. days |
| | baseline | | | |

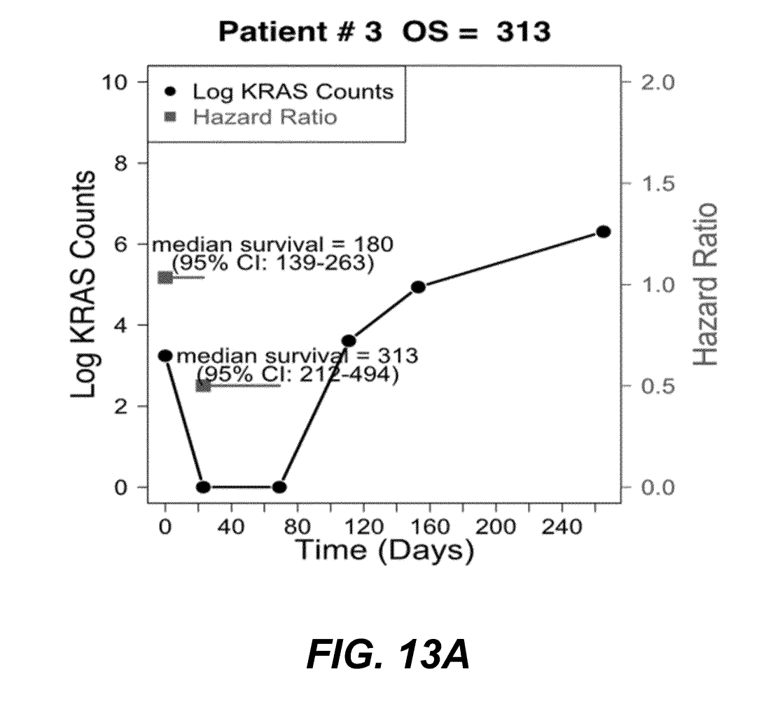

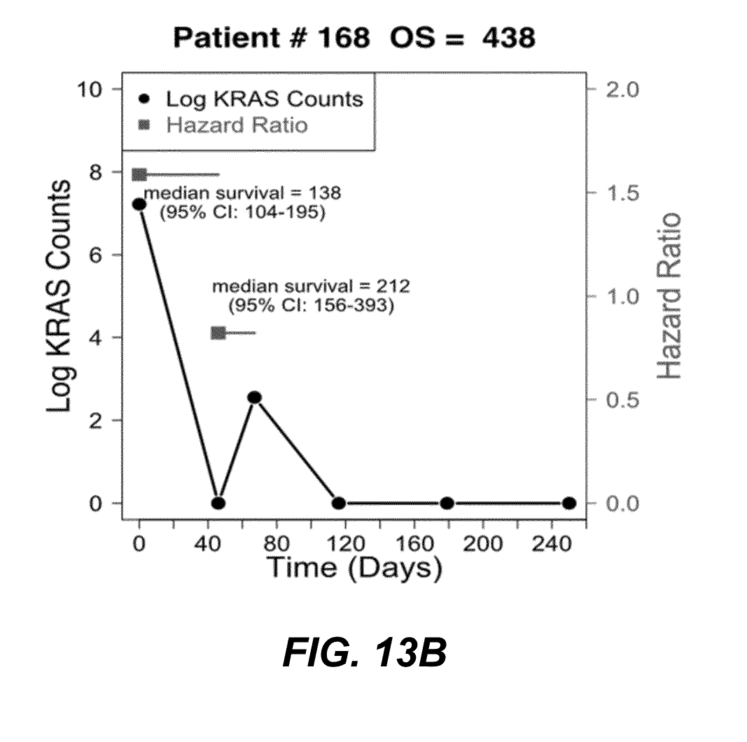

FIG. 13 provides plots of KRAS G12/13 counts over time and hazard ratios relative to a patient with ≦5.5 cps/10⁵ geq KRAS G12/13 at baseline (FIG. 13A) and a patient with >5.5 cps/10⁵ at baseline (FIG. 13B). Estimated and actual patient survival is shown.

When taking into account ctDNA KRAS levels after 2 weeks on treatment, an estimated median survival more accurately reflects actual survival of individual patients (as compared to the median survival estimated based on pre-treatment ctDNA KRAS levels only).

### Conclusions

In a study of 182 patients with locally advanced or metastatic pancreatic cancer, a statistically significant negative association was found between baseline ctDNA KRAS counts and OS (p<0.0001). A combination of ctDNA KRAS and CA 19-9 was a more powerful predictor of OS than either marker alone and allowed identification of a group of patients (17%) with significantly greater overall survival.

Use of the time-dependent model for monitoring patients beyond baseline allows more accurate assessment of responsiveness to therapy and associated increase or decrease of predicted survival.

### REFERENCES

Badalian-Very et al., 2010, Blood 116:1919-23.
Benesova et al., 2013, Anal Biochem. 433:227-34.
Brose et al., 2002, Cancer Res 62:6997-7000.
Curtin et al., 2005, N Engl J Med 353:2135-47.
Davies et al., 2002, Nature 417:949-54.
Dawson et al., 2013, N Engl J Med. 368:1199-1209.
Diehl et al., 2008, Nat Med. 14:985-990.
Forshew et al., 2012, Science Translational Medicine, 4:136ra168.
Keshet Y and Seger R., 2010, Methods Mol Biol. 661:3-38.
Niault T and Baccarini M., 2010, Carcinogenesis. 31:1165-74.
Puxeddu et al., 2004, J Clin Endocrinol Metab 89:2414-20.
Shaw et al., 2012, Genome Res. 22:220-31.
Tie et al., 2011, Int J Cancer 128:2075-84.
Wan et al., 2004, Cell 116:855-67.
Wellbrock C and Hurlstone A, 2010, Pharmacol. 80:561-7.
U.S. Pat. No. 8,623,603.
US Patent Application Publication US2010/0068711.
U.S. Provisional Patent Application No. 62/039,905.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

What is claimed is:

1. A method of determining responsiveness to a treatment or overall survival in a patient with a cancer undergoing the treatment, wherein the cancer is associated with a gene mutation, the method comprising
(a) obtaining a baseline sample of a bodily fluid, wherein the baseline sample was taken from the patient at the start of the treatment;
(b) quantitatively or semi-quantitatively determining the amount of the mutation in cell free DNA (cfDNA) in the baseline sample;
(c) obtaining a second ample of the bodily fluid, wherein the second sample was taken from the patient within about one month after the start of the treatment;
(d) quantitatively or semi-quantitatively determining the amount of the mutation in cell free DNA (cfDNA) in the second sample; and
(e) determining the responsiveness to the treatment by evaluating the results obtained in step (b) and/or step (d) by a predetermined criteria.

2. The method of claim 1, wherein the second sample is taken from the patient at about two weeks after the start of the treatment.

3. The method of claim 1, wherein the bodily fluid is serum or plasma.

4. The method or claim 1, wherein the bodily fluid is urine.

5. The method of claim 1, wherein the mutation is in a ABL1, BRAF, CHEK1, FANCC, GATA3, JAK2, MITF, PDCD1LG2, RBM10, STAT4, ABL2, BRCA1, CHEK2, FANCD2, GATA4, JAK3, MLH1, PDGFRA, RET, STK11, ACVR1B, BRCA2, CIC, FANCE, GATA6, JUN, MPL, PDGFRB, RICTOR, SUFU, AKT1, BRD4, CREBBP, FANCF, GID4(C17orf39), KAT6A (MYST3), MRE11A, PDK1, RNF43, SYK, AKT2, BRIP1, CRKL, FANCG, GLI1, KDM5A, MSH2, PIK3C2B, ROS1, TAF1, AKT3, BTG1, CRLF2, FANCL, GNA11, KDM5C, MSH6, PIK3CA, RPTOR, TBX3, ALK, BTK, CSF1R, FAS, GNA13, KDM6A, MTOR, PIK3CB, RUNX1, TERC, AMER1 (FAM123B), C11orf30 (EMSY), CTCF, FAT1, GNAQ, KDR, MUTYH, PIK3CG, RUNX1T1, TERT promoter, APC, CARD11, CTNNA1, FBXW7, GNAS, KEAP1, MYC, PIK3R1, SDHA, TET2, AR, CBFB, CTNNB1, FGF10, GPR124, KEL, MYCL (MYCL1), PIK3R2, SDHB, TGFBR2, ARAF, CBL, CUL3, FGF14, GRIN2A, KIT, MYCN, PLCG2, SDHC, TNFAIP3, ARFRP1, CCND1, CYLD, FGF19, GRM,3 KLHL6, MYD88, PMS2, SDHD, TNFRSF14, ARID1A, CCND2, DAXX, FGF23, GSK3B, KMT2A (MLL), NF1, POLD1, SETD2, TOP1, ARID1B, CCND3, DDR2, FGF3, H3F3A, KMT2C (MLL3), NF2, POLE, SF3B1, TOP2A, ARID2, CCNE1, DICER1, FGF4, HGF, KMT2D (MLL2), NFE2L2, PPP2R1A, SLIT2, TP53, ASXL1, CD274, DNMT3A, FGF6, HNF1A, KRAS, NFKBIA, PRDM1, SMAD2, TSC1, ATM, CD79A, DOT1L, FGFR1, HRAS, LMO1, NKX2-1, PREX2, SMAD3, TSC2, ATR, CD79B, EGFR, FGFR2, HSD3B1, LRP1B, NOTCH1, PRKAR1A, SMAD4, TSHR, ATRX, CDC73, EP300, FGFR3, HSP9OAA1, LYN, NOTCH2, PRKCI, SMARCA4, U2AF1, AURKA, CDH1, EPHA3, FGFR4, IDH1, LZTR1, NOTCH3, PRKDC, SMARCB1, VEGFA, AURKB, CDK12, EPHA5, FH, IDH2, MAGI2, NPM1, PRSS8, SMO, VHL, AXIN1, CDK4, EPHA7, FLCN, IGF1R, MAP2K1, NRAS, PTCH1, SNCAIP, WISP3, AXL, CDK6, EPHB1, FLT1, IGF2, MAP2K2, NSD1, PTEN, SOCS1, WT1, BAP1, CDK8, ERBB2, FLT3, IKBKE, MAP2K4, NTRK1, PTPN11, SOX10, XPO1, BARD1, CDKN1A, ERBB3, FLT4, IKZF1, MAP3K1, NTRK2, QKI, SOX2, ZBTB2, BCL2, CDKN1B, ERBB4, FOXL2, IL7R, MCL1, NTRK3, RAC1, SOX9, ZNF217, BCL2L1, CDKN2A, ERG, FOXP1, INHBA, MDM2, NUP93, RAD50, SPEN, ZNF703, BCL2L2, CDKN2B, ERRFI1, FRS2, INPP4B, MDM4, PAK3, RAD51, SPOP, BCL6, CDKN2C, ESR1, FUBP1, IRF2, MED12, PALB2, RAF1, SPTA1, BCOR, CEBPA, EZH2, GABRA6, IRF4, MEF2B, PARK2, RANBP2, SRC, BCORL1, CHD2, FAM46C, GATA1, IRS2, MEN1, PAX5, RARA, STAG2, BLM, CHD4, FANCA, GATA2, JAK1, MET, PBRM1, RB1, or STAT3 gene.

6. The method of claim 1, wherein the mutation is in the BRAF gene.

7. The method of claim 6, wherein the BRAF gene mutation is V600E.

8. The method of claim 1, wherein the mutation is in the KRAS gene.

9. The method of claim 8, wherein the mutation is KRAS G12A, G12C, G12D, G12R, G12S, G12V or G13D.

10. The method of claim 1, wherein overall survival is determined in a patient with pancreatic cancer.

11. The method of claim 10, further comprising determining CA 19-9 antigen levels.

12. The method of claim 1, wherein responsiveness to treatment for colorectal cancer is determined by evaluating results from both step (b) and step (d).

13. The method of claim 12, wherein the colorectal cancer is metastatic colorectal cancer.

14. The method of claim 12, wherein the gene mutation is a KRAS codon 12 or 13 mutation.

15. The method of claim 1, wherein the predetermined criteria is established by comparing treatment outcomes with the amount of the mutation in baseline samples and/or second samples in previous cancer patients.

16. The method of claim 15, wherein the treatment outcomes is overall survival of the previous cancer patients.

17. The method of claim 1, wherein a third sample of a bodily fluid is taken after the second sample, and the amount of the mutation in cell free DNA (cfDNA) in the third sample is quantitatively or semi-quantitatively determined.

18. The method of claim 1, wherein the patient has not previously undergone testing for the mutation.

19. The method of claim 12, wherein if responsiveness is determined, recommending continuing with the treatment, and if non-responsiveness is determined, recommending changing treatment.

20. The method of claim 12, wherein if responsiveness is determined, continuing with the treatment, and if non-responsiveness is determined, changing treatment.
